(19) United States
(12) Patent Application Publication
(10) Pub. No.: US 2019/0014996 A1
(43) Pub. Date: Jan. 17, 2019

(54) SMART MEDICAL DIAGNOSIS SYSTEM

(71) Applicants: Hai Rong Qian, Markham (CA); Jin Zhang, Shanghai (CN)

(72) Inventors: Hai Rong Qian, Markham (CA); Jin Zhang, Shanghai (CN)

(21) Appl. No.: 15/650,738

(22) Filed: Jul. 14, 2017

(51) Int. Cl. A61B 5/021; A61B 1/24; A61B 5/00

(52) U.S. Cl. CPC A61B 5/02108; A61B 1/24; A61B 5/0022; A61B 5/6898; A61B 5/0013

(57) ABSTRACT

A system for medically diagnosing a user includes at least one wearable device, an attachment means, a set of sensors, a controller, an electronic device, a device camera, a device display, a device controller, a device memory, a set of cloud-based servers and a software application. The system for medically diagnosing a user is useful for medical diagnosis using a smartphone application and wearable devices.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of medical diagnosis means and more specifically relates to a software application and wearable device for performing medical diagnosis.

### 2. Description of Related Art

Traditional Chinese Medicine (TCM) is a style of traditional medicine informed by modern medicine but built on a foundation of more than 2,500 years of Chinese history. One of the basic tenets of TCM holds that the body's vital energy (chi or qi) circulates through channels called meridians that have branches connected to bodily organs and functions. TCM's view of the body places little emphasis on anatomical structures, but is mainly concerned with the identification of functional entities (which regulate digestion, breathing, aging etc.). While health is perceived as the harmonious interaction of these entities and the outside world, disease is interpreted as a disharmony in interaction. TCM diagnosis aims to trace symptoms to patterns of an underlying disharmony, by measuring the pulse, inspecting the tongue, skin, and eyes, and looking at the eating and sleeping habits of the person as well as many other things.

To diagnose a patient, TCM practitioners examine human body parts such as the tongue, the smell of the breath, and strength of pulses on the pulse points of the body to initiate a therapy. Since the discovery of TCM, examination of the human tongue and the pulses of the heart have continued to be the primary sources of diagnostic information for determining health conditions when applying TCM. Devices such as blood pressure monitors, oximeters, smart watches, and even smartphones have been developed and are known to measure pulse rate. However, simply using these devices will not give a user any information as to what the pulse rate means for them in terms of TCM. Further, a full diagnosis for TCM involves visit to a doctor that specializes in TCM diagnosis. This is costly and time consuming, especially if the doctor is unable to perform pulse diagnosis. Thus, a suitable solution is desired.

U.S. Pat/Pub. No. 2017/0071481 to Be Xie relates to a wearable device for pulse reading. The described wearable device for pulse reading includes a smart pulse reading device placed on a patient's hand or wrist, with which a pulse reading technique through digitalization and signal processing enables telemedicine such that a patient can receive medical attention. The invention includes the benefit of generating a specific prescription for a patient depending on the medical diagnosis. One of the prescriptions generated, may be traditional Chinese medicine used as an alternate form of treatment. By accurately assessing the patient's pulses, the smart device can determine the type of medical condition the patient is affected by. The device features various sensors that are placed above a critical radial artery in the wrist of a patient, and embedded in the device are the pressure sensors in a matrix orientation. The matrix of sensors can detect the pressure of the patient's pulses by calculating the pressure change over time.

### BRIEF SUMMARY OF THE INVENTION

In view of the foregoing disadvantages inherent in the known medical diagnosis means art, the present disclosure provides a novel smart medical diagnosis system. The general purpose of the present disclosure, which will be described subsequently in greater detail, is to provide a software application and wearable device to medically diagnosis a user.

A system for medically diagnosing a user is disclosed herein. The system for medically diagnosing a user may include at least one wearable device configured for attachment to the at least one pulse point. The at least one wearable device may include attachment means configured to secure the at least one wearable device to the at least one pulse point; a set of sensors fixedly attached to the wearable device, the set of sensors may be configured to contact the at least one pulse point when the at least one wearable device is attached to the at least one pulse point, and further may be configured to measure a pulse of the user and produce a plurality of pulse-signals based on the measure of the pulse; and a controller disposed within the at least one wearable device. The controller may include wireless communication means, and be in electronic communication with the set of sensors. Further, the controller may be configured to receive and transmit the plurality of pulse-signals from the set of sensors.

The system for medically diagnosing a user may further include an electronic device remote from the wearable device and including: a device camera configured to capture an image of the tongue; a device display; a device controller configured to wirelessly communicate with the wireless communication means and further configured to receive the plurality of pulse-signals from the controller; a device memory configured to store a plurality of medical parameter information files and a plurality of medical diagnosis information files; a set of cloud-based servers configured to store the plurality of medical parameter information files and the plurality of medical diagnosis information files; and a software application installed on the electronic device and in communication with the device camera, the device display, the device memory and the device controller.

A method of using the system for medically diagnosing a user is also disclosed herein. The method of using system for medically diagnosing a user may comprise the steps of: providing the system for medically diagnosing a user as above; wirelessly connecting the wearable device with the electronic device; attaching the at least one wearable device to the at least one pulse point; opening the software application installed on the electronic device; capturing an image of the tongue with the device camera; and reviewing a medical diagnosis information file chosen by the software application and presented on the device display. Further steps may include: saving the medical diagnosis information file chosen by the software application to a profile located on the set of cloud-based servers; and selectively reviewing the profile within the software application.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a smart medical diagnosis system, constructed and operative according to the teachings of the present disclosure.

FIG. 1 is a front perspective view of the system for medically diagnosing a user during an ‘in-use’ condition, according to an embodiment of the disclosure.

FIG. 2 is a front perspective view of the system for medically diagnosing a user of FIG. 1, illustrating at least one wearable device and a pulse-point according to an embodiment of the present disclosure.

FIG. 3 is a front perspective view of the system for medically diagnosing a user of FIG. 1, illustrating wireless communication between the at least one wearable device and an electronic device, according to an embodiment of the present disclosure.

FIG. 4 is a front perspective view of the system for medically diagnosing a user of FIG. 1, illustrating a smartphone application installed on the electronic device, according to an embodiment of the present disclosure.

FIG. 5 is a flow diagram illustrating a method of use for system for medically diagnosing a user, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a medical diagnosis means and more particularly to a smart medical diagnosis system as used to improve the medical diagnosis using a software application and wearable devices.

Generally disclosed is a pair of wearable devices. One may be installed on each wrist of a user. Each unit may comprise three pulse sensors that are placed at designated spots on the wrist. The readings from the sensors may be tabled and compared to diagnose the health conditions of various internal organs (intestine, spleen, bladder, heart, liver, kidney, stomach, lung and gall bladder). A conclusion of the overall health of the user may then be reported.

The wearable devices may be controlled by a smartphone application, which may display the results and suggestions in a user-friendly manner. The app also may keep a record of all user health conditions for long term personalized health advice. An image of a tongue may be taken with the smart phone for TCM tongue diagnosis. The image may then be compared to existing data on the server (color, shape, texture, moisture, coating, thickness, cracks). The image and the corresponding results may be added to the collection of data.

The application may track, monitor and even predict user's health condition—provide timely suggestions/warning. Further, the application may find similarities in the data pattern for research of cure and prevention of difficult diseases (e.g., we may discover certain pulse patterns and/or tongue patterns for patients with Meniere's disease).

The smart medical diagnosis system may be offered to doctors who offer TCM, and to consumers who may use it to conveniently check their health conditions, keep track of their health conditions and get advice on how to improve their health conditions. Smart medical diagnosis system may further host a network of TCM doctors that can take appointments with the users to do a remote, or in-person video consultation.

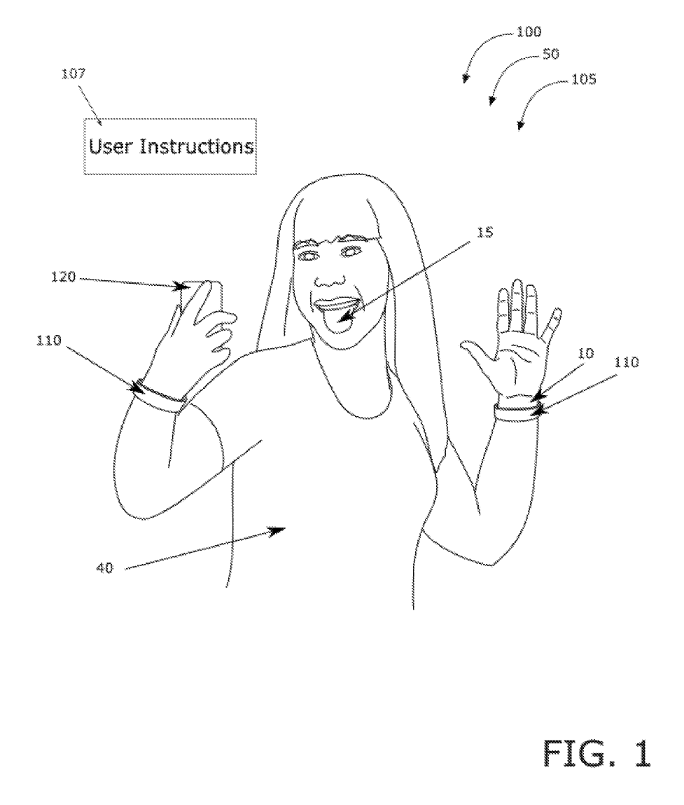

Referring now more specifically to the drawings by numerals of reference, there is shown in FIGS. 1-5, various views of a system for medically diagnosing a user (“system” 100).

FIG. 1 shows a system 100 during an ‘in-use’ condition 150, according to an embodiment of the present disclosure. As illustrated, the system 100 may be used by the user 40 having at least one pulse-point 10 and a tongue 15. The system 100 may include at least one wearable device 110, an attachment means 112, a set of sensors 114, a controller 116, an electronic device 120, a device camera 122, a device display 124, a device controller 126, a device memory 128, a set of cloud-based servers 142, and a software application 130. As shown in the figure, the device camera 122 may be configured to capture an image of the tongue 15.

According to one embodiment, the system 100 may be arranged as a kit. The kit may include a set of user instructions 107. The instructions 107 may detail functional relationships in relation to the structure of the system 100 (such that the system 100 can be used, maintained, or the like, in a preferred manner).

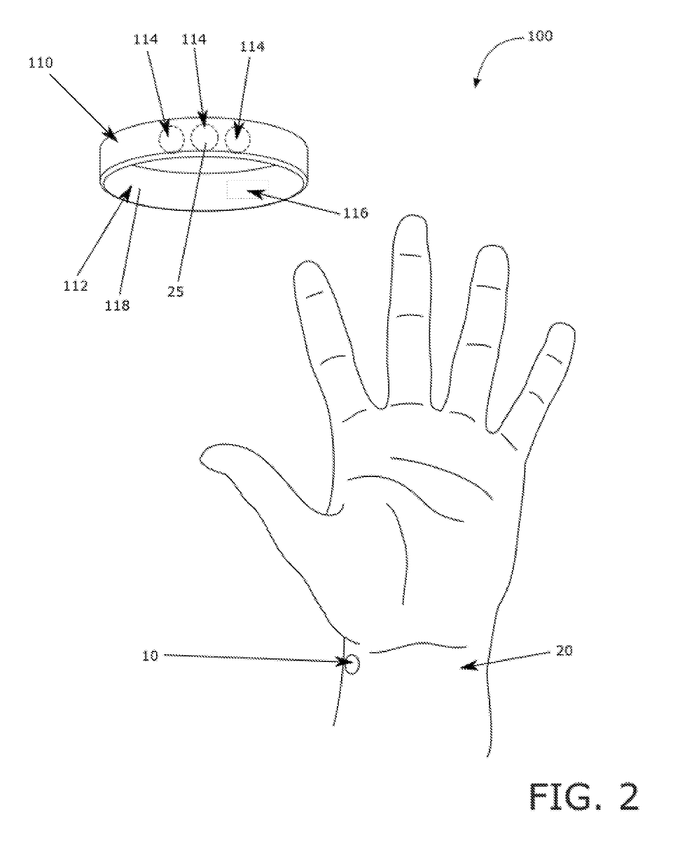

FIG. 2 shows a front perspective view of the system 100 for medically diagnosing a user 40 of FIG. 1, according to an embodiment of the present disclosure. Illustrated here is the at least one wearable device 110, the set of sensors 114 and a wrist 20 of the user 40 illustrating the at least one pulse-point 10. The at least one wearable device 110 may be configured for attachment to the at least one pulse-point 10 and include the attachment means 112 configured to secure the at least one wearable device 110 to the at least one pulse-point 10. Further, the at least one wearable device 110 may include the set of sensors 114 fixedly attached to the wearable device 110; the set of sensors 114 may be configured to contact the at least one pulse-point 10 when the at least one wearable device 110 is attached to the at least one pulse-point 10, and further may be configured to measure a pulse of the user 40 and produce a plurality of pulse-signals based on the measure of the pulse. In addition, the controller 116 may be disposed within the at least one wearable device 110. The controller 116 may include wireless communication means in electronic communication with the set of sensors 114 and may be configured to receive and transmit the plurality of pulse-signals from the set of sensors 114. In one embodiment, the attachment means 112 may be a wrist strap 118.

In a preferred embodiment, the at least one wearable device 110 may be two wearable devices 110, and the at least one pulse-point 10 may be the wrist 20. Further, the set of sensors 114 may be three sensors and the three sensors may be placed at designated areas 25 on the wrist 20. In this preferred embodiment, the designated areas 25 may be Cun (first position), Guan (second position), and Chi (third position). The Guan location may be indicated to the user 40 via a red circle located on the wearable devices 110. This location may be found opposite the styloid process of the radius. The two wearable devices 110 may be marked as “left” and “right”. Further, the two wearable devices 110 may include a small display screen demonstrating basic status information.

The set of sensors 114 may be configured to apply a plurality of pressures on the pulse point 10. The plurality of pressures may vary from light pressure, medium pressure and heavy pressure. During pulse-diagnosis, strength of a pulse may be measured during the light pressure, medium pressure and heavy pressure phases. Differences between the strength of the pulse during each phase may be measured and may create the plurality of pulse-signals. Similarly, a position of the pulse may be measured during the light pressure, medium pressure and heavy pressure phases. Differences between the position during each may be again measured, creating the plurality of pulse-signals. Likewise, a rhythm of the pulse may be measured during the light pressure, medium pressure and heavy pressure phases. In a further example, a speed of the pulse may be measured.

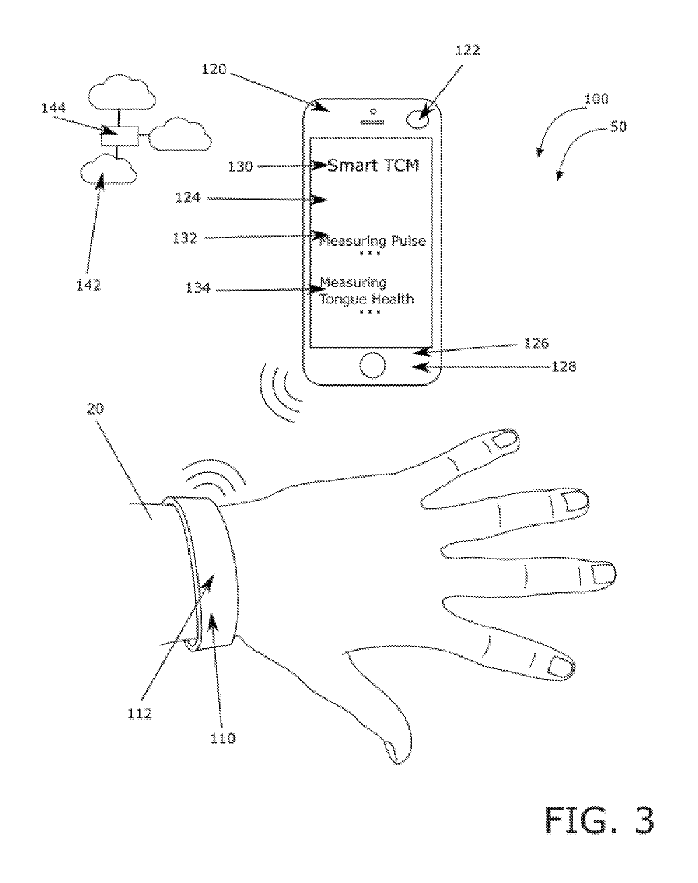

FIG. 3 shows a front perspective view of the system 100 for medically diagnosing a user 40 of FIG. 1, according to an embodiment of the present disclosure. Illustrated here is the at least one wearable device 110 attached to the at least one pulse-point 10 of the user's 40 wrist 20 and the electronic device 120 demonstrating the smartphone application 130. Further illustrated is wireless communication between the wearable device 110 and the electronic device 120.

The electronic device 120 may be remote from the wearable device 110. The electronic device 120 may include the device camera 122 configured to capture the image of the tongue 15, the device display 124, the device controller 126 which may be configured to wirelessly communicate with the wireless communication means and further configured to receive the plurality of pulse-signals from the controller 116, the device memory 128 which may be configured to store a plurality of medical parameter information files and a plurality of medical diagnosis information files; and the software application 130 which may installed on the electronic device 120 and in communication with the device camera 122, the device display 124, the device memory 128 and the device controller 126. The wireless communication means may be by chosen from Wi-Fi, BLUETOOTH® (wireless communication means comprises a wireless technology for exchanging data over short distances using short-wavelength UHF radio waves in the ISM band from 2.4 to 2.485 GHz.), Zigbee, and any future wireless technologies. In the preferred embodiment, the electronic device 120 may be a smartphone. In other examples, the electronic device 120 may be a tablet computer, a laptop, or the like.

In the preferred embodiment, the system 100 may further include the set of cloud-based servers 142. The set of cloud-based servers 142 may include a cloud storage-unit 144 configured to store the plurality of medical parameter information files and the plurality of medical diagnosis information files. In this embodiment, the electronic device 120, the at least one wearable device 110 and the software application 130 may be in communication with the set of cloud-based servers 142 and the cloud storage-unit 144. Information pertaining to the plurality of pulse-signals and the image of the tongue 15 may also be received and stored on the cloud storage unit 144. Further, the set of cloud-based servers 142 may be configured to process information relating to the plurality of medical parameter information files and the plurality of medical diagnosis information files.

The software application 130 may be configured to receive the image of the tongue 15 and the plurality of pulse-signals. Further, the software application 130 may be configured to choose one of the medical diagnosis information files based on a comparison of the medical parameter information files, and the image of the tongue 15 and the plurality of pulse-signals. In addition, the software application 130 may be further configured to display the medical diagnosis information file on the device display 124.

The medical parameter information files may include tongue-information 134 and pulse-information 132. In a preferred embodiment, the tongue-information 134 may be images of tongues in various conditions. In this embodiment, the conditions may relate to tongue-color, tongue-shape, tongue-texture, tongue-moisture, tongue-coating, tongue-thickness, and tongue-cracks. Further, the pulse-information 132 may include pulse-strength, pulse-speed and pulse-rhythm.

In a preferred embodiment, the medical diagnosis information files may include the tongue-information 134 and the pulse-information 132 as they relate to the health condition of various internal organs 5. The various internal organs 5 may be intestines, spleen, bladder, heart, liver, kidney, stomach, lung and gall bladder. As an example, a tongue-information 134 file may be a tongue-shape with swollen sides, and a related medical diagnosis information file may indicate that the tongue-shape with swollen sides is associated with poor health condition of the liver. As a further example, a tongue-information 134 file may be a tongue-coating that is thin and white in color. A related medical diagnosis information file may indicate that the tongue-coating that is thin and white in color is associated with good health condition of the various internal organs. An example relating to pulse-information 132, may include a pulse-information 132 file which may be a pulse-rhythm that is intermittent, and a medical diagnosis information file may indicate that the pulse-rhythm that is intermittent may be associated with poor health condition of the heart.

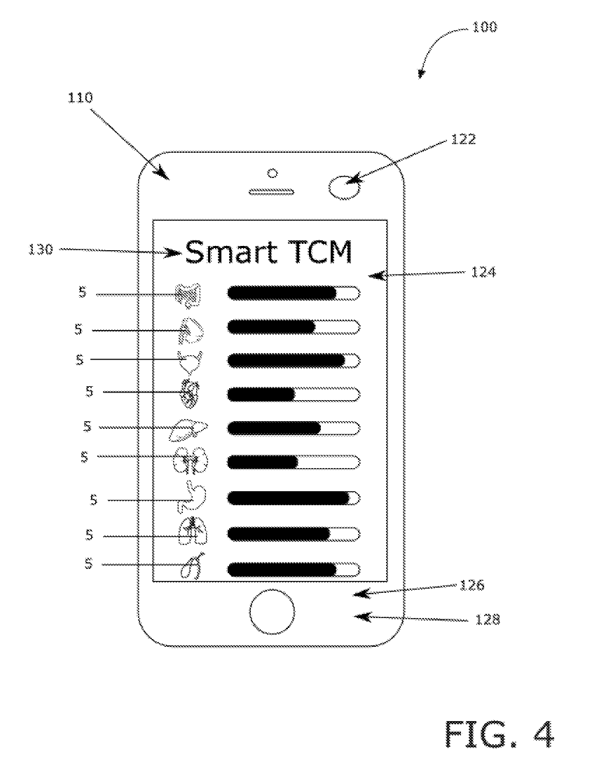

FIG. 4 shows a front perspective view of the system 100 for medically diagnosing a user 40 of FIG. 1, according to an embodiment of the present disclosure. Illustrated here is the smartphone application 130 having chosen a medical diagnosis information file and indicating overall health condition of the various internal organs 5. As above, the software application 130 may be configured to choose one of the medical diagnosis information files based on a comparison of the medical parameter information files, and the image of the tongue 15 and the plurality of pulse-signals. In addition, the software application 130 may be further configured to display the medical diagnosis information file on the device display 124. Other tangible results may pertain such as the printing off of results and the like.

The device memory 128 may be configured to store a profile-information of the user 40. The profile-information may be the image of the tongue 15, the plurality of pulse-signals and the medical diagnosis information file chosen by the software application 130. In addition, the smartphone application may further be configured to produce push-notifications on the electronic device 120 relating to the medical diagnosis information files. In the preferred embodiment, the software application 130 may be configured to track, monitor and predict the user's 40 health conditions. In this embodiment, the push-notifications may relate to suggestions or warnings to alert the user 40 of the predictions. As above, this information may be stored on the cloud storage-unit 144, or alternatively, stored on the device memory 128.

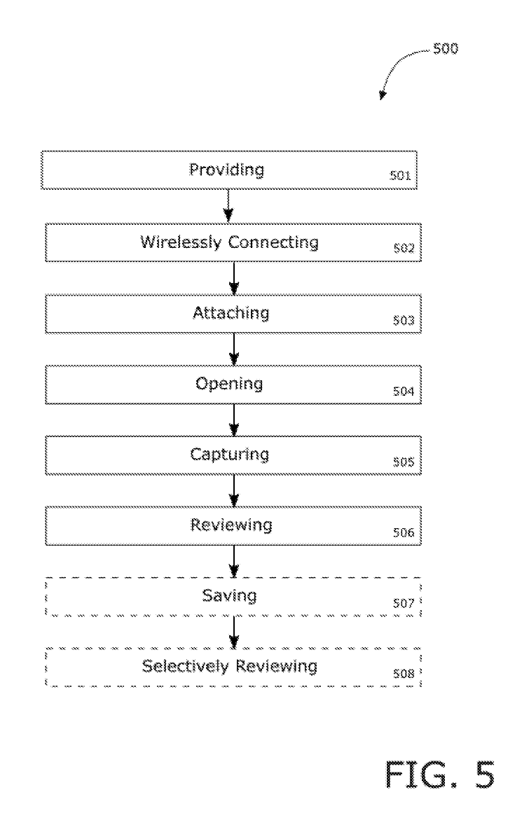

FIG. 5 is a flow diagram illustrating a method 500 for using a system 100 to medically diagnose a user 40, according to an embodiment of the present disclosure. As illustrated, the method 500 for using a system 100 to medically diagnose a user 40 may include the steps of: step one 501, providing the system 100 as above; step two 502, wirelessly connecting the wearable device 110 with the electronic device 120; step three 503, attaching the at least one wearable device 110 to the at least one pulse-point 10; step four 504, opening the software application 130 installed on the electronic device 120; step five 505, capturing an image of the tongue 15 with the device camera 122; and step six 506, reviewing a medical diagnosis information file chosen by the software application 130 and presented on the device display 124. Further steps may include: step seven 507, saving the medical diagnosis information file chosen by the software application 130 to a profile located on the set of cloud-based servers 142; and step eight 508, selectively reviewing the profile within the software application 130.

It should be noted that step seven 507 and step eight 508 are optional steps and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for system 100 for medically diagnosing a user 40 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

1. A system for medically diagnosing a user, the user having at least one pulse point and a tongue, the system comprising:
at least one wearable device configured for attachment to the at least one pulse point, the at least one wearable device including:
attachment means configured to secure the at least one wearable device to the at least one pulse point;
a set of sensors fixedly attached to the wearable device, the set of sensors configured to contact the at least one pulse point when the at least one wearable device is attached to the at least one pulse point, and further configured to measure a pulse of the user and produce a plurality of pulse-signals based on the measure of the pulse; and
a controller disposed within the at least one wearable device, the controller including wireless communication means, the controller being in electronic communication with the set of sensors and configured to receive and transmit the plurality of pulse-signals from the set of sensors; and
an electronic device remote from the wearable device, the electronic device including:
a device camera configured to capture an image of the tongue;
a device display;
a device controller configured to wirelessly communicate with the wireless communication means and further configured to receive the plurality of plurality of pulse-signals from the controller;
a device memory configured to store a plurality of medical parameter information files and a plurality of medical diagnosis information files;
a set of cloud-based servers configured to store the plurality of medical parameter information files and the plurality of medical diagnosis information files; and
a software application installed on the electronic device and in communication with the device camera, the device display, the device memory and the device controller;
wherein the software application is configured to receive the image of the tongue and the plurality of pulse-signals;
wherein the software application is further configured to choose one of the medical diagnosis information files based on a comparison of the medical parameter information files, and the image of the tongue and the plurality of pulse-signals; and
wherein the software application is further configured to display the medical diagnosis information file on the device display.

2. The system of claim 1, wherein the medical parameter information files include tongue-information and pulse-information.

3. The system of claim 2, wherein the tongue-information is images of said tongue(s) in various conditions.

4. The system of claim 3, wherein the conditions relate to tongue-color, tongue-shape, tongue-texture, tongue-moisture, tongue-coating, tongue-thickness, and tongue-cracks.

5. The system of claim 2, wherein the pulse information is pulse-strength, pulse-speed and pulse-rhythm.

6. The system of claim 5, wherein the medical diagnosis information files include the tongue-information and the pulse-information as they relate to the health condition of various internal organs.

7. The system of claim 6, wherein the various internal organs are intestines, spleen, bladder, heart, liver, kidney, stomach, lung and gall bladder.

8. The system of claim 1, wherein the device memory and the set of cloud-based servers are further configured to store the profile-information of the user.

9. The system of claim 8, wherein the profile-information is the image of the tongue, the plurality of pulse-signals and the medical diagnosis information file chosen by the software application.

10. The system of claim 1, wherein the smartphone application is further configured to produce push-notifications on the electronic device relating to the medical diagnosis information files.

11. The system of claim 1, wherein the at least one wearable device is two wearable devices.

12. The system of claim 1, wherein the at least one pulse point is a wrist.

13. The system of claim 12, wherein the set of cloud-based servers are further configured to process information relating to the plurality of medical parameter information files and the plurality of medical diagnosis information files.

14. The system of claim 12, wherein the set of sensors comprise exactly three said sensors.

15. The system of claim 14, wherein the set of sensors are configured to apply a plurality of pressures on the pulse point.

16. The system of claim 1, wherein the wireless communication means comprises a wireless technology for exchanging data over short distances using short-wavelength UHF radio waves in the ISM band from 2.4 to 2.485 GHz.

17. A system for medically diagnosing a user, the user having at least one pulse point and a tongue, the system comprising:
at least one wearable device configured for attachment to the at least one pulse point, the at least one wearable device including:
attachment means configured to secure the at least one wearable device to the at least one pulse point;
a set of sensors fixedly attached to the wearable device, the set of sensors configured to contact the at least one pulse point when the at least one wearable device is attached to the at least one pulse point, and further configured to measure a pulse of the user and produce a plurality of pulse-signals based on the measure of the pulse; and
a controller disposed within the at least one wearable device, the controller including wireless communication means, the controller being in electronic communication with the set of sensors and configured to receive and transmit the plurality of pulse-signals from the set of sensors; and
an electronic device remote from the wearable device, the electronic device including:
a device camera configured to capture an image of the tongue;
a device display;
a device controller configured to wirelessly communicate with the wireless communication means and further configured to receive the plurality of pulse-signals from the controller;
a device memory configured to store a plurality of medical parameter information files and a plurality of medical diagnosis information files;
a set of cloud-based servers configured to store the plurality of medical parameter information files and the plurality of medical diagnosis information files; and
a software application installed on the electronic device and in communication with the device camera, the device display, the device memory and the device controller;
wherein the software application is configured to receive the image of the tongue and the plurality of pulse-signals;
wherein the software application is further configured to choose one of the medical diagnosis information files based on a comparison of the medical parameter information files, and the image of the tongue and the plurality of pulse-signals;
wherein the software application is further configured to display the medical diagnosis information file on the device display;
wherein the medical parameter information files include tongue-information and pulse-information;
wherein the tongue-information is images of tongues in various conditions;
wherein the conditions relate to tongue-color, tongue-shape, tongue-texture, tongue-moisture, tongue-coating, tongue-thickness, and tongue-cracks;
wherein the pulse information is pulse-strength, pulse-speed and pulse-rhythm;
wherein the medical diagnosis information files include the tongue-information and the pulse-information as they relate to the health condition of various internal organs;
wherein the various internal organs are intestines, spleen, bladder, heart, liver, kidney, stomach, lung and gall bladder;
wherein the device memory and the set of cloud-based servers are further configured to store the profile-information of the user;
wherein the profile-information is the image of the tongue, the plurality of pulse-signals and the medical diagnosis information file chosen by the software application;
wherein the smartphone application is further configured to produce push-notifications on the electronic device relating to the medical diagnosis information files;
wherein the at least one wearable device is two wearable devices;
wherein the at least one pulse point is a wrist;
wherein the set of cloud-based servers are further configured to process information relating to the plurality of medical parameter information files and the plurality of medical diagnosis information files;
wherein the set of sensors are three sensors;
wherein the set of sensors are configured to apply a plurality of pressures on the pulse point; and
wherein the wireless communication means is wireless communication means comprises a wireless technology for exchanging data over short distances using short-wavelength UHF radio waves in the ISM band from 2.4 to 2.485 GHz.

18. The system of claim 17, further comprising set of instructions; and
wherein the system is arranged as a kit.

19. A method for using a system to medically diagnose a user, the user having at least one pulse point and a tongue, the method comprising the steps of:
providing at least one wearable device including:
attachment means configured to secure the at least one wearable device to the at least one pulse point, a set of sensors fixedly attached to the wearable device, the set of sensors configured to contact the at least one pulse point when the at least one wearable device is attached to the at least one pulse point, and further configured to measure a pulse of the user and produce a plurality of pulse-signals based on the measure of the pulse, and a controller disposed within the at least one wearable device, the controller including wireless communication means, the controller being in electronic communication with the set of sensors and configured to receive and transmit the plurality of pulse-signals from the set of sensors;
providing an electronic device remote from the wearable device, the electronic device including:
a device camera configured to capture an image of the tongue, a device display, a device controller configured to wirelessly communicate with the wireless communication means and further configured to receive the plurality of pulse-signals from the controller, a device memory configured to store a plurality of medical parameter information files and a plurality of medical diagnosis information files; a set of cloud-based servers configured to store the plurality of medical parameter information files and the plurality of medical diagnosis information files; and a software application installed on the electronic device and in communication with the device camera, the device display, the device memory and the device controller;
wirelessly connecting the wearable device with the electronic device;
attaching the at least one wearable device to the at least one pulse point;
opening the software application installed on the electronic device;
capturing an image of the tongue with the device camera; and
reviewing a medical diagnosis information file chosen by the software application and presented on the device display.

20. The method of claim 19, further comprising the steps of:
saving the medical diagnosis information file chosen by the software application to a profile within the software application; and
selectively reviewing the profile within the software application.
